(19) United States
(12) Patent Application Publication
(10) Pub. No.: US 2021/0308296 A1
(43) Pub. Date: Oct. 7, 2021

(54) SANITIZING CABINET FOR BANK TELLERS

(71) Applicant: Cook Security Group Inc., Portland, OR (US)

(72) Inventors: Brian COOK, Vancouver, WA (US); Daniel SMALLWOOD, Ridgefield, WA (US)

(21) Appl. No.: 17/129,512

(22) Filed: Dec. 21, 2020

Related U.S. Application Data
(60) Provisional application No. 63/003,703, filed on Apr. 1, 2020.
(60) Provisional application No. 63/010,360, filed on Apr. 15, 2020.
(60) Provisional application No. 63/026,650, filed on May 18, 2020.
(60) Provisional application No. 63/033,719, filed on Jun. 2, 2020.

(51) Int. Cl. A61L 2/10; A47B 49/00; A61L 2/26

(52) U.S. Cl. CPC A61L 2/10; A47B 49/008; A61L 2/26; A61L 2202/11; A61L 2202/122; A61L 2202/121; A47B 2220/0077

(57) ABSTRACT

An ultraviolet light (UV) sanitizing cabinet for banking equipment may include one or more UV light assemblies coupled to an electronics module and disposed within a housing. In some examples, the UV sanitizing cabinet may include racks or compartments configured to receive pneumatic tube canisters. In some examples, the UV sanitizing cabinet may include rollers or tumblers configured to rotate the pneumatic tube canisters to allow UV sanitization of all sides of the canisters.

### CROSS-REFERENCES

The following applications and materials are incorporated herein, in their entireties, for all purposes: U.S. Provisional Patent Application Ser. No. 63/003,703, filed Apr. 1, 2020; U.S. Provisional Patent Application Ser. No. 63/010,360, filed Apr. 15, 2020; U.S. Provisional Patent Application Ser. No. 63/026,650, filed May 18, 2020; and U.S. Provisional Patent Application Ser. No. 63/033,719, filed Jun. 2, 2020.

### FIELD

This disclosure relates to systems and methods for sanitizing small items, such as casino- and banking-related equipment. More specifically, the disclosed embodiments relate to systems and methods for using ultraviolet (UV) light to disinfect small objects.

### INTRODUCTION

Given the nature of casino and bank teller services, a significant amount of interaction with the public is frequently required. Equipment such as ATMs and pneumatic tube systems are often used to facilitate this interaction, for example, during drive-up services. Moreover, small items such as currency and gaming chips often change hands. Due to the myriad of transmissible and/or infectious diseases, good health practices and sanitizing equipment are needed to keep workers and customers safe. This is especially true in light of events such as the COVID-19 pandemic.

### SUMMARY

The present disclosure provides systems, apparatuses, and methods relating to UV sanitizing cabinets.

In some embodiments, a sanitizing cabinet includes: a cabinet housing including one or more walls defining an interior space; an ultraviolet (UV) light-emitting device coupled to the cabinet housing such that the UV light-emitting device is configured to emit UV light into the interior space; and a receiving apparatus configured to securely hold a canister within the interior space in a position exposed to the UV light.

In some embodiments, a sanitizing system includes: a cabinet including a cabinet housing defining a compartment; an ultraviolet (UV) radiation-producing device coupled to the cabinet; an electronic controller coupled to the UV radiation-producing device and configured to control the UV radiation-producing device to selectively emit ultraviolet radiation into the compartment; a canister having at least one canister wall that is transmissive with respect to ultraviolet radiation; and a canister support structure disposed within the compartment and configured to receive the canister, such that contents of the canister are exposed to the ultraviolet radiation from the UV radiation-producing device through the canister wall.

In some embodiments, a method for sanitizing small objects includes: opening an access door of a cabinet having an ultraviolet (UV) lamp housed therein; placing a canister into a canister receiving structure of the cabinet through the access door, wherein the canister contains an object to be sanitized; closing the access door and, using the UV lamp, irradiating the object in the canister with UV light for a selected duration, wherein the object is exposed to the UV light through a wall of the canister; and increasing exposure of the object to the UV light by automatically reorienting the canister during the selected duration.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is an isometric view of a first illustrative UV sanitizing cabinet in accordance with aspects of the present disclosure.

FIG. 2 is an isometric view of the UV sanitizing cabinet of FIG. 1, shown with a top panel removed.

FIG. 3 is an isometric view of a first illustrative canister rack for use in the UV sanitizing cabinet of FIG. 1.

FIG. 4 is an isometric view of a second illustrative canister rack for use in the UV sanitizing cabinet of FIG. 2.

FIG. 5 is an isometric view of a second illustrative UV sanitizing cabinet in accordance with aspects of the present disclosure.

FIG. 6 is a partial isometric view of a compartment of the UV sanitizing cabinet of FIG. 5.

FIG. 7 is a front isometric view of an illustrative pneumatic tube canister, in accordance with aspects of the present disclosure.

FIG. 8 is a top schematic view of an illustrative UV sanitizing drawer in accordance with aspects of the present disclosure.

FIG. 9 is a side schematic view of the UV sanitizing drawer of FIG. 8.

FIG. 10 is an isometric view of a third illustrative UV sanitizing cabinet in accordance with aspects of the present disclosure.

FIG. 11 is a front schematic view of the UV sanitizing cabinet of FIG. 10.

FIG. 12 is an isometric view of a fourth illustrative UV sanitizing cabinet in accordance with aspects of the present disclosure.

FIG. 13 is a front schematic view of the UV sanitizing cabinet of FIG. 12.

FIG. 14 is a flow chart depicting steps of an illustrative method for sanitizing potentially contaminated objects using UV light according to aspects of the present teachings.

FIG. 15 is a schematic diagram of an illustrative data processing system as described herein.

FIG. 16 is a schematic diagram of an illustrative network data processing system as described herein.

FIG. 17 is a partial front isometric view of a fifth illustrative UV sanitizing cabinet in accordance with aspects of the present disclosure.

FIG. 18 is an isometric view of a mesh drum configured to be utilized in the sanitizing cabinet of FIG. 17.

### DETAILED DESCRIPTION

Various aspects and examples of a UV sanitizing cabinet having containers, racks, and/or shelves configured to hold canisters (e.g., pneumatic tube canisters or the like) and/or other items, as well as related methods, are described below and illustrated in the associated drawings. Unless otherwise specified, a UV sanitizing cabinet in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Elongate” or “elongated” refers to an object or aperture that has a length greater than its own width, although the width need not be uniform. For example, an elongate slot may be elliptical or stadium-shaped, and an elongate candlestick may have a height greater than its tapering diameter. As a negative example, a circular aperture would not be considered an elongate aperture.

“Coupled” means connected, either permanently or releasably, whether directly or indirectly through intervening components.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

A “controller” or “electronic controller” includes processing logic programmed with instructions to carry out a controlling function with respect to a control element. For example, an electronic controller may be configured to receive an input signal, compare the input signal to a selected control value or setpoint value, and determine an output signal to a control element (e.g., a motor or actuator) to provide corrective action based on the comparison. In another example, an electronic controller may be configured to interface between a host device (e.g., a desktop computer, a mainframe, etc.) and a peripheral device (e.g., a memory device, an input/output device, etc.) to control and/or monitor input and output signals to and from the peripheral device.

Directional terms such as “up,” “down,” “vertical,” “horizontal,” and the like should be understood in the context of the particular object in question. For example, an object may be oriented around defined X, Y, and Z axes. In those examples, the X-Y plane will define horizontal, with up being defined as the positive Z direction and down being defined as the negative Z direction.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

The ultraviolet (UV) portion of the electromagnetic radiation spectrum falls between visible light and x-ray radiation. Within the UV portion of the spectrum, light having shorter wavelengths (e.g., 200-300 nanometers (nm)) is referred to as UVC or UV-C. UVC light is germicidal, as exposure to UVC light typically results in the eradication or inactivation of exposed bacteria and viruses. Sources of UVC light include, e.g., UVC LED lamps, mercury-based lamps, continuous-output xenon lamps, pulsed-xenon lamps, other suitable gas-discharge lamps, and/or any other suitable sources.

Germicidal effects of UVC light are dependent on a dosage of UVC light delivered to a specific object or surface. An increase in time and/or intensity of UVC light delivered to an object or surface generally corresponds to a greater decrease in microbes (e.g., viruses, bacteria, fungi, etc.) present on the object or surface. UVC light is effective for treating chemical-resistant microbes, as well as in applications where use of chemical disinfectants may be unsafe or otherwise undesirable.

In general, a UV sanitizing cabinet in accordance with aspects of the present teachings may include an enclosure having an access door, one or more sources of ultraviolet light mounted within and/or adjacent the enclosure, and one or more internal storage racks, shelves, or devices configured to hold and/or automatically reorient small items such as canisters (AKA carriers) in position(s) suitable for being irradiated by the UV light source(s).

In some examples, a UV sanitizing cabinet is sized and/or otherwise configured to fit under a typical casino or bank teller countertop, and may therefore be referred to as an under-counter cabinet.

In some examples, a conversion kit is provided to transform an existing cabinet into a UV sanitizing cabinet, e.g., by replacing an upper drawer assembly with a UV light assembly.

In some examples, the UV sanitizing cabinet includes integrated canisters or other containers, which may be configured to hold small items such as coins or poker chips. In some examples, the UV sanitizing cabinet includes canister reorientation (e.g., rotation) mechanisms configured to facilitate improved UV exposure of a canister and/or its contents.

UV sanitizing cabinets in accordance with the present disclosure may include one or more other suitable features, such as an electronic controller and/or other suitable control system configured to control the UV light source and/or to control a user interface (UI) to present information to the user.

Aspects of a control system for UV sanitizing cabinets of the present teachings may be embodied as a computer method, computer system, or computer program product. Accordingly, aspects of the electronic controller may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the electronic controller may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the electronic controller may be written in one or any combination of programming languages, including an object-oriented programming language (such as Java, C++), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the electronic controller may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the electronic controller. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved.

Each block and/or combination of blocks may be implemented by special purpose hardware-based systems (or combinations of special purpose hardware and computer instructions) that perform the specified functions or acts.

### Examples, Components, and Alternatives

The following sections describe selected aspects of illustrative UV sanitizing cabinets as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. First Illustrative UV Sanitizing Cabinet

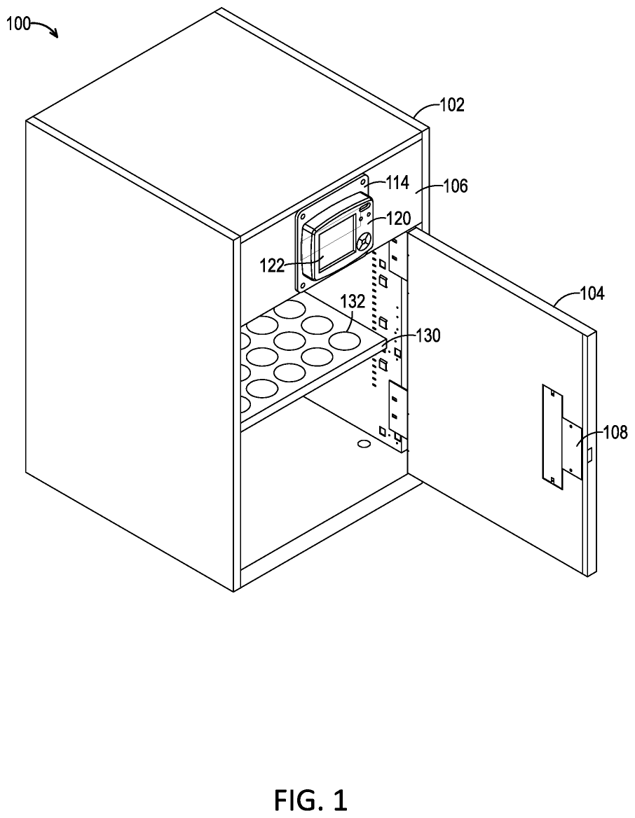

As shown in FIGS. 1-4, this section describes an illustrative UV sanitizing cabinet 100. Cabinet 100 is an example of the cabinets described in the Overview above.

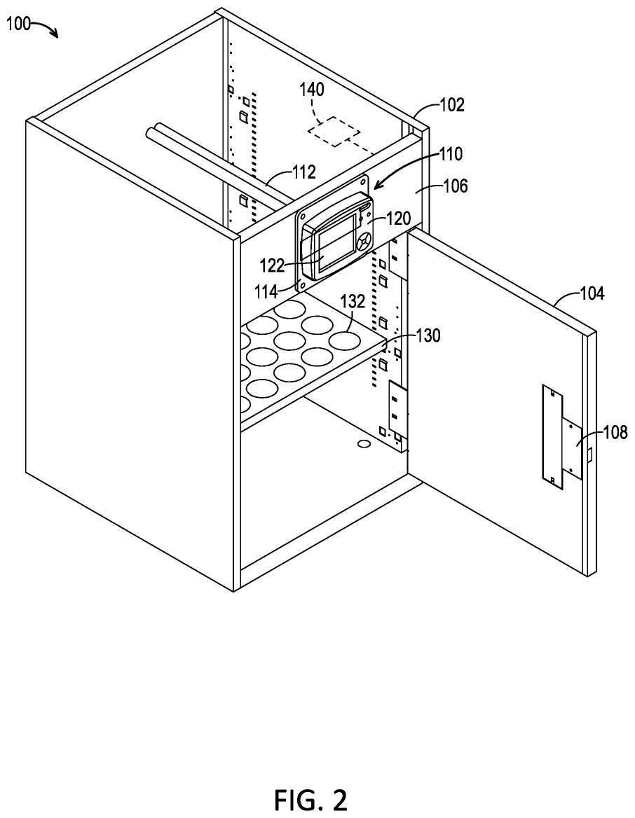

As depicted in FIGS. 1 and 2, cabinet 100 includes a generally cuboidal enclosure 102 having an access door 104 coupled to a front face by side hinges, such that the enclosure and door define an internal space or volume (AKA an interior) of the cabinet. In this example, enclosure 102 has walls, a floor, and a ceiling (AKA panels) that are all generally planar. The enclosure and door comprise a rigid structural material (e.g., steel or plastic) that is opaque to UV light (e.g., preventing transmission of UV light, such that people and objects outside of the cabinet are protected from the UV radiation produced within the cabinet). In some examples, cabinet 100 may be sized to fit beneath a counter, such as a counter at a bank teller window.

Door 104 is shorter (i.e., has a smaller height) than the overall face of the enclosure, and a panel 106 is disposed above the door to complete the enclosure. A UV light assembly 110 is coupled to the cabinet at panel 106, such that one or more UV light elements 112 protrude (e.g., through panel 106) into an upper portion of the interior of the cabinet. UV light elements 112 may comprise any suitable device(s) configured to emit light having suitable UV wavelength(s). Although elements 112 are referred to herein as UV light elements, the elements may in some examples also emit light having other wavelengths (e.g., in the visible portion of the spectrum) in addition to the UV light.

Light assembly 110 is secured in place by a mounting plate 114 coupling the light assembly to panel 106. A human-machine interface (HMI) 120 of the light assembly is disposed on an outer surface of the mounting plate, such that the HMI is accessible to a user when door 104 is closed.

In some examples, a locking mechanism 108 is disposed on an interior surface of door 104. Locking mechanism 108 may comprise any suitable electrically and/or mechanically actuatable locking system, such as one or more electromagnets, latches, tumblers, and/or the like.

Light assembly 110 comprises an electronics module 122 (disposed within the cabinet, outside the cabinet, or a combination thereof) coupled to one or more of the UV light elements 112 (in this case, a pair), configured to illuminate and/or irradiate the interior of the cabinet. In some examples, a reflector is disposed near the light elements (e.g., between the light elements and one or more cabinet panels) to increase the amount of illumination reaching a desired portion of the interior. In some examples, an interior lining, whether continuous, piecewise continuous, or discrete, is included on at least one inner wall of the enclosure, and configured to reflect UV light internally to increase germicidal effectiveness. For example, interior walls of the cabinet and/or rack(s) 130 may comprise aluminum, aluminum paneling, aluminum coatings (e.g., paint), and/or aluminum linings (e.g., foil). Additionally, or alternatively, light assembly 110 may be disposed in a different suitable location (e.g., at top, bottom, sides, back, front of the interior space). One or more additional UV light assemblies may be present in any suitable location(s).

Electronics module 122 may include any suitable electronic controller(s) configured to monitor and/or control aspects of the UV light elements and/or the overall UV light assembly generally, and/or to display related information (e.g., via HMI 120). For example, electronics module 122 may include an electronic controller coupled to one or more sensors (e.g., a door open/closed sensor), and/or HMI 120 described above. In some examples, electronics module 122 includes a wireless and/or wired transmitter, receiver, and/or transceiver for remote monitoring.

The electronic controller may be configured to control operation of the UV cabinet in any suitable manner. In some examples, a preset, programmable, and/or selectable timer is utilized to turn off the UV light after a given amount of time, and/or to alert the user to do so. In some examples, the door has a switch and/or other sensor to tell the electronic controller to turn the light off when the door is opened. In some examples, objects to be placed into the cabinet for sanitization are automatically identifiable by the electronic controller, e.g., using an RFID system 140. For example, the controller may use an RFID system to sense how many and what types of objects are in the cabinet, and in response, to set the timer and/or intensity of the light accordingly. See discussion of suitable RFID systems below. In some examples, the controller is configured to recognize objects in another suitable manner (e.g., using barcodes, QR codes, one or more camera(s) and computer vision techniques, and/or any other suitable methods),

Sanitizing effectiveness of UVC light is dosage-dependent. As UV intensity and exposure time (AKA duration) increase, disinfection effectiveness increases. For example, a UV sanitizing cabinet including a pair of UV light elements (as described above) may require a certain UV duration to effect a 99% decrease in microbes (or specific type(s) of microbes) on a given surface. In some examples, a user of the UV sanitizing cabinet may input a desired UV dose using HMI 120. A dose may be defined and/or characterized by a concentration of UV light (e.g., an energy per unit area in, e.g., J/m², a power per unit area, an energy or power per solid angle or per volume, and/or any other suitable characterization of an amount of UV light), a relative or absolute decrease in microbes (e.g., a log reduction in microbe quantity), a sanitizing time (e.g., exposure time), a spectrum and/or peak wavelength of emitted radiation, any other suitable measure, and/or any combination thereof.

Electronics module 122 may control the intensity and duration of UV light element(s) 112 to provide the desired dose. In some examples, electronics module 122 sets a desired UV dose in response to information received from RFID system 140. In some examples, electronics module 122 automatically locks the UV sanitizing cabinet using locking mechanism 108, so that items may not be removed from the cabinet before the desired UV dose is provided. In some examples, electronics module 122 tracks an element life of UV light element(s) 112 and notifies a user if one or more elements should be replaced. In some examples, electronics module 122 tracks a total sanitizing time of the cabinet.

One or more racks or shelves 130 may be permanently, removably, and/or adjustably installed within the interior of enclosure 102. Racks 130 may include any suitable support surface(s) or feature(s) configured to support and/or hold selected items in the interior space, such that the supported items are illuminated and therefore able to be sterilized by the UV light emitted by the UV light assembly.

Examples of racks 130 are depicted in FIGS. 1, 2, 3, and 4. In FIGS. 1 and 2, at least one rack 130 is a planar sheet having a plurality of holes or apertures 132 formed therein. Holes or apertures 132 may be sized and/or configured to receive elongate or cylindrical items typically utilized by casino or bank tellers, such as pens, rolls of coins, canisters for gaming chips, and/or pneumatic tube canisters.

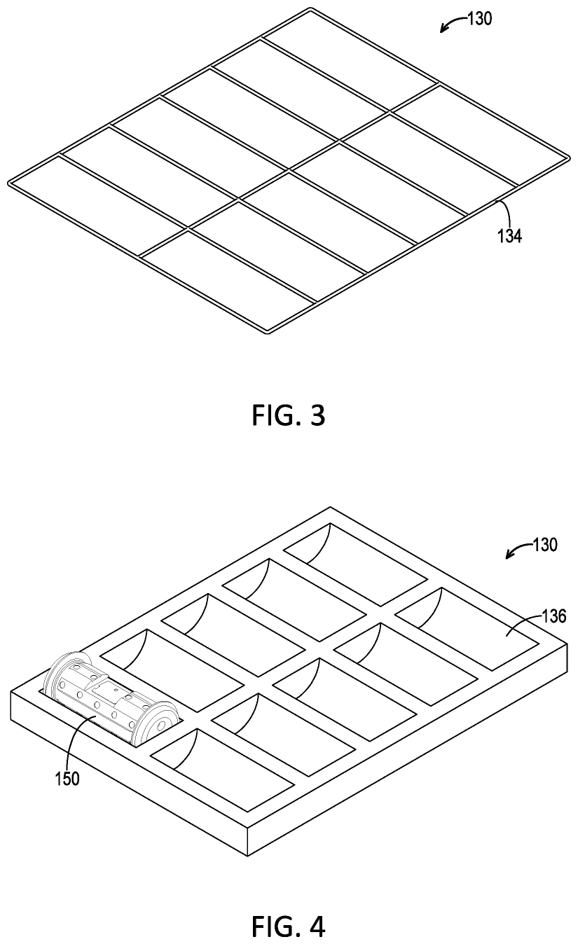

FIG. 3 depicts another example of rack 130, showing a wire rack having large rectangular spaces between smaller wires, tubes, and/or other suitable structural members 134 arranged in a grid pattern. In the rack of FIG. 3, elongate objects such as canisters can rest in the rectangular openings, either in a closed position or an opened position depending on the user's preference.

In FIG. 4, a third example of rack 130 is depicted. Recesses 136 are formed in a body of the rack, configured to receive objects therein. In this example, recesses 136 are semi-cylindrical and configured to receive cylindrical objects, such as canister 150.

In some examples, racks 130 may comprise any suitable material(s) configured to facilitate or increase application of UV light (e.g., UVC light) to an object held by the rack (e.g., by reflecting UV light toward the object, by allowing transmission of UV light through the rack onto the object, etc.). Example materials include aluminum, UV-transparent or transmissive plastic (e.g., fluorinated ethylene propylene (FEP) plastic), and/or any other suitable materials. FEP, for example, may have a transmittance of 90% or more with respect to UV light. In some examples, UV-transmissive materials having other properties are utilized, e.g., having UV transmittance of at least 25% or at least 50%.

### B. Second Illustrative UV Sanitizing Cabinet

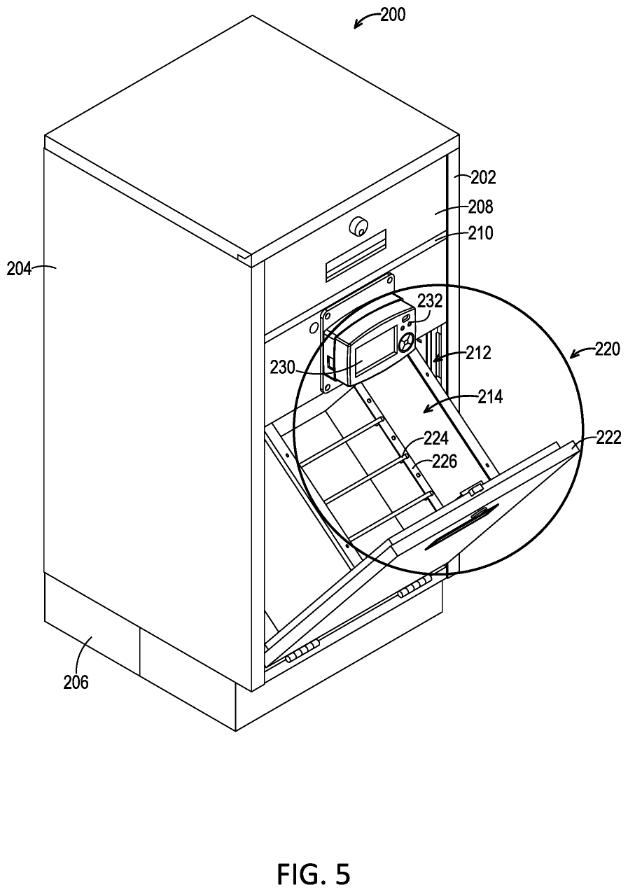

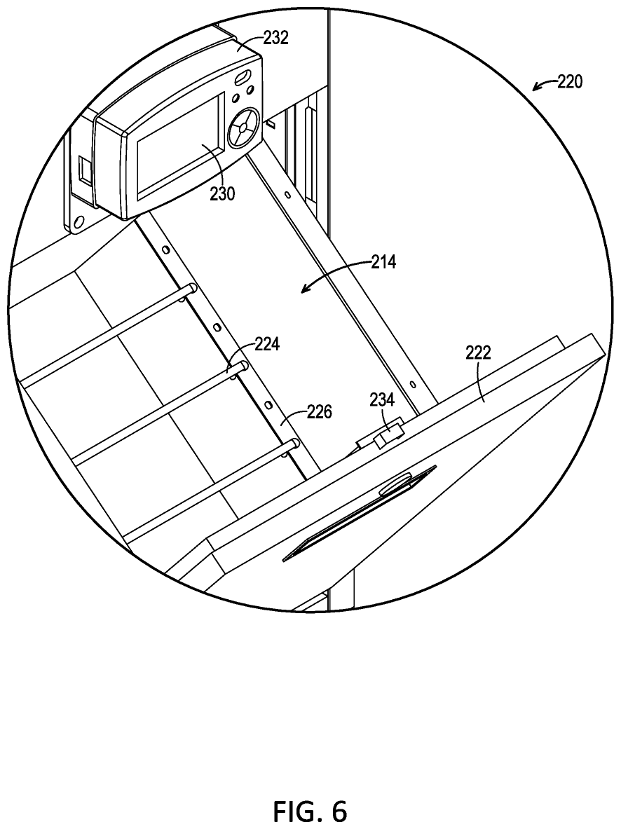

A second example of a UV sanitizing cabinet according to the present teachings is depicted in FIGS. 5 and 6. Illustrative cabinet 200 may include the same or similar general functionality and construction as cabinet 100, in at least some respects. Features of cabinet 200 may be combined with features of cabinet 100, as desired.

Cabinet 200 includes an outer housing 202 having a plurality of panels 204 and a base 206. A drawer 208 is slidingly disposed in an upper portion of the housing, and a divider 210 separates the drawer portion from the interior 212 of the cabinet.

In some examples, drawer 208 is a standard drawer (e.g., configured to perform ordinary functions of a bank teller's cabinet). In some examples, drawer 208 includes its own UV light source configured to irradiate object(s) placed in the drawer, which may allow objects inserted into drawer 208 to receive a different UV dose from objects disposed in other portions of the cabinet. Alternatively, or additionally, drawer 208 may have a transparent or perforated bottom, such that radiation emitted by a UV source disposed elsewhere in the cabinet can penetrate an interior of the drawer.

In some examples, drawer 208 has a lock. For example, the drawer lock may be controlled at least in part by a timer, preventing a user from opening the drawer until a selected exposure time has elapsed. The selected exposure time may be different from an exposure time of object(s) being sanitized elsewhere in the cabinet. In some examples, drawer 208 may be configured to receive a canister sanitizer (e.g., modular or permanent) as described below (e.g., in section D).

A tilt-out partial storage container 220 is selectively housed in cabinet interior 212. Container 220 includes sidewalls, a floor, and a front panel 222 defining a compartment 214. Front panel 222 is hinged to the cabinet at a lower edge of the front panel, such that container 220 can be tilted out of the cabinet by pivoting the front panel about the hinge. When container 220 has been tilted out of the cabinet, an at least partially open top of compartment 214 is accessible to a user. When container 220 is positioned within the cabinet, an interior of the compartment is illuminated by one or more UV light emitters (not shown) disposed within the cabinet (e.g., extending from and/or through a front wall of the cabinet into the cabinet interior, below drawer 208), as explained above with respect to cabinet 100. In some examples, container 220 has no floor of its own, and functions as a tilt-out door with sidewalls.

Compartment 214 includes one or more internal dividers. The one or more internal dividers may include a configurable set of rods 224 releasably coupled at either end to rails 226. In this example, a pair of rails 226 are disposed along sidewalls of container 220 within compartment 214, but in general rails and/or other suitable devices for mounting the rods may be disposed in any suitable location(s).

In this example, one of the rails 226 has aperture(s) facing upward (e.g., toward a top of container 220), and the other rail has aperture(s) facing sideways (e.g., facing the other rail). Rods 224 are L-shaped, having one end fitting into a sideways-facing aperture and another end fitting into an upward-facing aperture. In general, however, the rails and rods may have any suitable configuration(s).

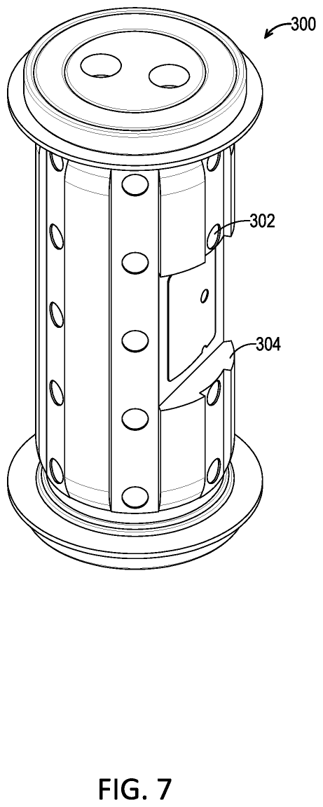

Compartment 214, rods 224, and other internal components may comprise any suitable material configured to reflect UV light, or in some examples, to be transparent to UV light (e.g., having relatively high UV transmission). For example, one or more reflective components may comprise aluminum. In some examples, rods 224, and/or other internal components comprise fluorinated ethylene propylene (FEP) plastic, which is highly transmissive with respect to visible and UV light. FEP, for example, may have a transmittance of 90% or more with respect to UV light. In some examples, UV-transmissive materials having other properties are utilized, e.g., having a UV transmittance of at least 25% or at least 50%. In some examples, carriers and/or canisters, such as those illustrated in FIG. 7 and described below, may comprise FEP, to facilitate sanitization of the carrier and assorted contents/components.

Storage container 220 is configured to tilt into and out of the interior of cabinet 200 to provide unfettered access to the upper end of the compartment, which is at least partially open-mouthed. In some examples, this arrangement also facilitates leaving the UV lamp in an “on” condition (e.g., emitting radiation) regardless of container position. For example, one or more sidewalls of cabinet 200 and/or a floor of container 220 may comprise a UV-blocking (i.e., opaque) material and/or otherwise shield the user from the UV light inside the interior of the cabinet when container 220 is in a tilted-out position. This may allow the UV lamp to continue sanitizing objects disposed within drawer 208 while container 220 is in a tilted-out position, in examples where the drawer floor allows transmission of UV radiation into the drawer interior.

In some examples, because storage container 220 facilitates insertion and removal of canisters while the UV source is in an “on” condition, e.g., by blocking the UV light with respect to the user in some or all positions of the container, one or more objects may be swapped out of the container without turning off the UVC lamp. In some examples, an electronics module 230 and/or human-machine interface (HMI) 232 individually tracks canisters or other objects inserted into the cabinet, and alerts a user when each canister is sanitized. Electronics module 230 and HMI 232 may be similar or identical to electronics module 122 and HMI 120, described above. In some examples, tracking the canisters may be facilitated by an RFID system, as described above and below.

In some examples, tilting storage container 220 out of the interior of cabinet 200 switches the UV light source from an “on” condition to an “off condition. Cabinet 200 may include a sensor configured to sense an orientation or position of container 220, which may instruct electronics module 230 to switch conditions of the UV light source. In some examples, the sensor is disposed adjacent to the lock of the cabinet, and is configured to sense whether the cabinet is locked or unlocked. In some examples, the sensor is disposed adjacent to a tilting mechanism of the storage container, and is configured to sense whether the container is in a vertical or tilted orientation.

In some examples, canisters may be supported by two or more rods 224. In some examples, a diameter of a canister suitable for use in cabinet 200 (e.g., canister 300) may be larger than a distance between two of rods 224. In some examples, canisters may be sized such that each space between two rods 224 may fit one canister disposed in a horizontal orientation. In some examples, cabinet 200 includes a lock 234 (disposed, e.g., on front panel 222 and/or any other suitable location), which may electronically controlled to prevent the user from opening the cabinet before a desired UV dose is administered (e.g., to one or multiple canisters).

In some examples, inserting a canister into compartment 214 may include removing one of rods 224, placing the canister into the compartment, and replacing the rod 224. In some examples, canisters may be inserted in a vertical orientation, such that a first end of the canister is disposed beneath the rods and a second end of the canister is disposed above the rods. In some examples, electronics module 230 may “lock” the rod in place once a desired UV dose is inputted by the user or designated by the electronics module, so that the canister may not be removed from the cabinet before the desired UV dose is administered.

### C. Illustrative Canister or Carrier

Turning now to FIG. 7, an illustrative canister or carrier 300 in accordance with aspects of the present teachings is depicted. Canister 300 is substantially similar to a standard bank teller canister, further comprising holes, apertures, and/or openings 302 formed in an outer shell 304. The holes are configured to permit UVC light from outside the carrier to pass into the interior so as to sanitize contents of the carrier. For example, a driver's license, gaming chips, bank card, currency, etc. may be sanitized in this manner.

One or more of the holes may be at least partially covered and/or filled with a UVC-transmissive material (e.g., FEP), or may be empty and uncovered, or some holes may be empty and some may be covered and/or filled to some extent. FEP, for example, may have a transmittance of 90% or more with respect to UV light. In some examples, UV-transmissive materials having other properties are utilized, e.g., having UV transmittance of at least 25% or at least 50%. Although round openings are depicted in FIG. 7, any suitable size, shape, and number of openings may be utilized. In some examples, some or all of the walls of the canister may comprise UVC-transmissive material, such as FEP and/or the like.

Canister 300 may include one or more RFID tags (disposed, e.g., on an interior or exterior surface, or embedded in the canister), which enable information about the canister to be transmitted and stored using an RFID system, as described above and below.

### D. Illustrative Canister Sanitizer

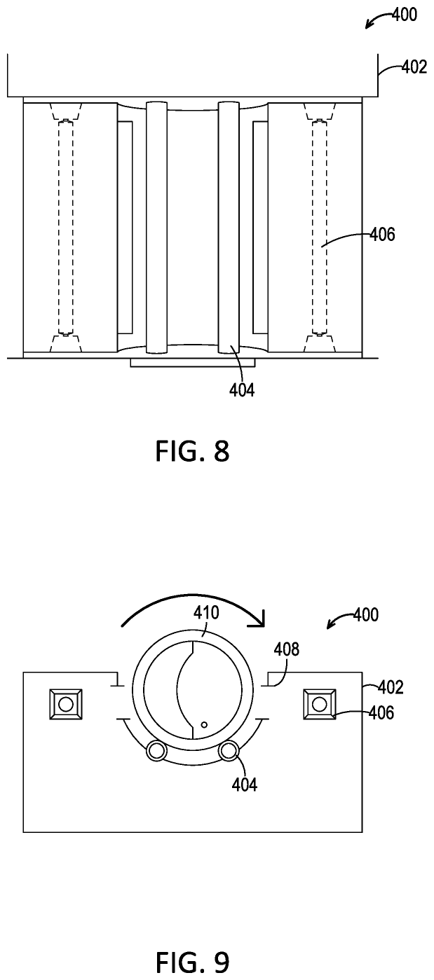

FIGS. 8 and 9 depict an illustrative canister sanitizing device 400. Device 400 may be configured to fit into a cabinet such as those depicted and described herein, e.g., in the top drawer, and to sanitize a carrier or canister such as the one depicted in FIG. 7.

Device 400 includes an enclosure or housing 402. Housing 402 includes a body (e.g., comprising metal) having a channel or recess sized to receive a carrier. A set of powered (e.g., motorized) rollers 404 are disposed within or adjacent the recess and are sized and configured to receive a carrier 410 thereon. Rollers 404 are configured to rotate the carrier (e.g., on its long axis). Rollers 404 may be powered by any suitable system for rotating rollers, including a direct-drive motor, one or more gearboxes or gearing systems, motorized belt or chain drives, etc.

UVC light emitters 406 (e.g., UVC lamps and/or any other suitable sources of UVC radiation) are disposed on one or both sides of rollers 404 in locations suitable for radiating UVC light onto the carrier when on the rollers (e.g., illuminating the interior of the carrier via openings within walls of the carrier housing, and/or through UVC-transparent portions of the carrier housing). In the example depicted in FIGS. 8 and 9, the UVC lamps are disposed within housing 402 on opposing sides of the recess, and are configured to provide light through openings 408 formed in the housing of the device (e.g., in walls of the recess). Disposing the lamps within the housing of the device facilitates limiting of UVC exposure to a user. In some examples, the walls of the recess additionally or alternatively comprise UVC-transparent portions allowing radiation from lamps 406 to reach the carrier.

In the depicted example, UVC emitters 406 comprise lamps disposed parallel to the recess. This arrangement allows radiation to be provided along a majority or entirety of the length of carrier 410 disposed within the recess. In some examples, emitters 406 comprise arrays of LEDs or other suitable device(s).

In some examples, UVC lamps 406 may be controlled using an electronics module, similar to the modules described above. In some examples, UVC lamps 406 are always on, or are controlled by the user using a switch or toggle disposed on an external surface of the housing. In some examples, device 400 is configured to be received within a drawer of the cabinet, such as drawers described above, and lamps 406 may be activated and deactivated in response to a signal from a drawer sensor, which indicates whether the drawer is open or closed.

In some examples, device 400 is modular. In some modular examples, device 400 includes data-communication connectors (e.g., USB ports and/or cables, HDMI ports and/or cables, etc.), which may be configured to connect UVC lamps 406 to an electronics module or HMI of a sanitizing cabinet, such as those described above and below.

In some examples, the light emitters are configured to rotate around the carrier instead of, or in addition to, the carrier rotating past the lights. In some examples, the carrier is held and rotated by one or both of its ends. In some examples, the carrier is enclosed in a generally cylindrical, stationary or moving array of lamps surrounding the carrier on all sides (e.g., lateral sides).

### E. Third Illustrative UV Sanitizing Cabinet

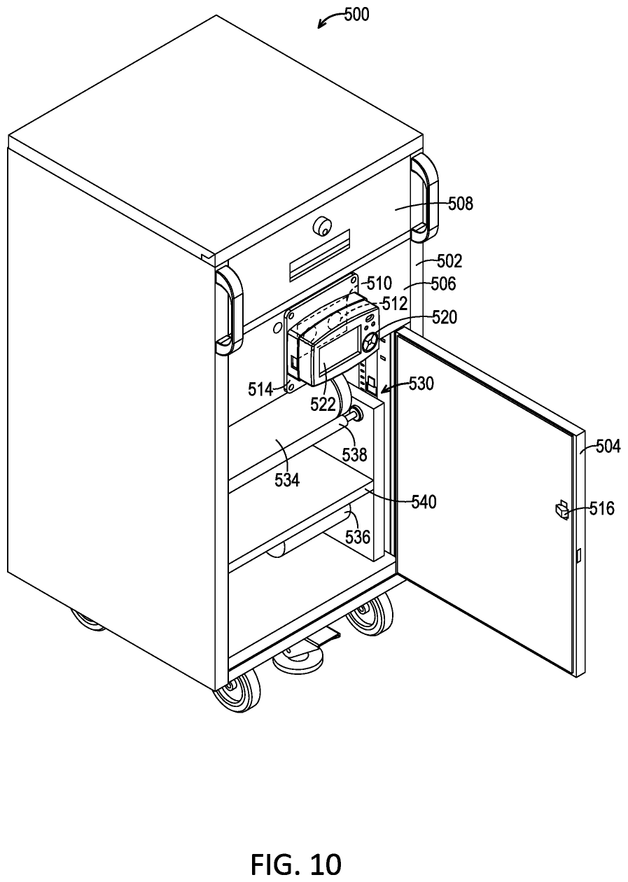

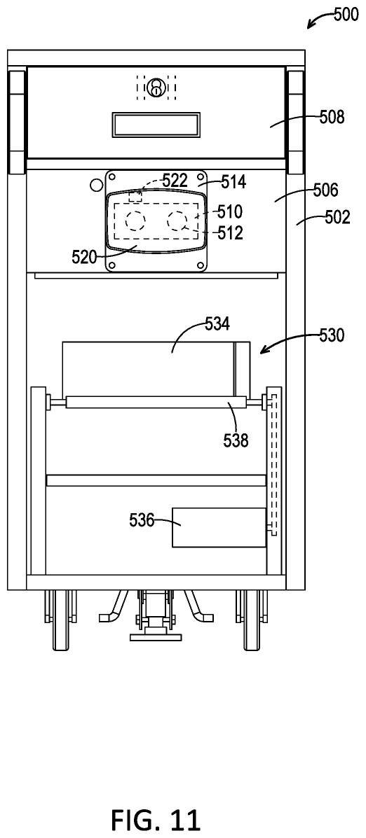

A third example of a UV sanitizing cabinet is depicted in FIGS. 10-11 and described below. Cabinet 500 may be substantially similar in at least some respects to cabinets 100 and 200, described above.

Cabinet 500 includes a generally cuboidal enclosure 502 having an access door 504 coupled to a front face by hinges (e.g., side hinges), such as those described above with respect to cabinet 100. Door 504 is shorter (i.e., has a smaller height) than the overall face of the enclosure, and a panel 506 is disposed on the front face between the door and a drawer 508 disposed at a top of the enclosure above panel 506. A UV light assembly 510 is coupled to the cabinet at panel 506, such that the UV light element(s) 512 protrude (e.g., through panel 506) into the upper portion of the interior of the cabinet. The light assembly is secured in place by a mounting plate 514, and a human-machine interface (HMI) 520 of the light assembly is disposed and accessible on an outer surface of the mounting plate. In some examples, a locking mechanism 516 is disposed on an interior surface of door 504. Locking mechanism 516 may comprise any suitable electrically and/or mechanically actuatable locking system, such as electromagnets, latches, tumblers, and/or the like.

The enclosure and door comprise a rigid structural material that is opaque to UV light, such as steel or plastic. In some examples, interior walls of the cabinet are at least partly reflective, and may comprise aluminum, aluminum paneling, aluminum coatings (e.g., paint), and/or aluminum linings (e.g., foil).

In some examples, drawer 508 is a standard drawer (e.g., with no particular sanitizing functionality). In some examples, drawer 508 includes its own UV light source. Alternatively, or additionally, drawer 508 may have a transparent and/or perforated bottom, such that light emitted by a source disposed elsewhere in the cabinet can penetrate an interior of the drawer. In some examples, drawer 508 is configured to receive a canister sanitizer (e.g., modular or permanent), such as device 400 described above.

Light assembly 510 includes an electronics module 522 coupled to one or more of the UV light elements 512 (in this case, a pair), configured to illuminate and/or irradiate the interior of the cabinet. Electronics module 522 may include any suitable electronic controller configured to monitor, control, and/or display information regarding the UV light elements and the overall UV light assembly generally. For example, electronics module 522 may include an electronic controller coupled to one or more sensors (e.g., a door open/closed sensor), and/or HMI 520 described above. In some examples, electronics module 522 includes a wireless or wired transmitter, receiver, and/or transceiver for remote monitoring. The electronic controller may be configured to control operation of the UV cabinet in any suitable manner, such as described above with respect to cabinet 100.

Cabinet 500 includes a casino chip sanitizing device 530 disposed in the interior of the cabinet. In the example depicted in FIGS. 10 and 11, chip sanitizing device 530 includes a plurality of rollers 538 which function to rotate a canister 534, the canister configured to contain a plurality of gaming chips, coins, tokens, and/or the like (e.g., as described above with respect to canister sanitizer 400). Rotating the canister provides a more even UV exposure for the canister, and may mechanically agitate the contents of the canister, thereby better exposing each item to the UV light. In some examples, canister 534 is partially or completely transparent to UV light, and/or are otherwise configured to allow UV light to illuminate canister contents (e.g., by including a plurality of apertures). In some examples, canister 534 is substantially similar to canister 300, described above. Additionally or alternatively, other types of canisters or containers can be rotated within the illuminated internal space of the cabinet using device 530.

Chip sanitizing device 530 includes a drive motor 536, in this case disposed beneath a shelf 540 of enclosure 502. In some examples, shelf 540 comprises a UV-blocking material and is configured to shield the motor from UV radiation. Motor 536 causes rotation of a canister drive feature of the chip sanitizing device, e.g., using belts, rollers, gears, chains, and/or any suitable combination thereof. Chip sanitizing device 530 includes one or more (e.g., a pair of parallel) rollers 538 driven by motor 536. In the example of FIG. 10, these rollers are disposed substantially parallel to a front surface of the cabinet enclosure, although other suitable orientations may be utilized, i.e., transverse to the front surface. Power may be transmitted from motor 536 to rollers 538 using any suitable transmission method, e.g., using a direct-drive, gearing, a gearbox, a belt or chain, and/or the like. Cabinet 500 is configured to receive one or more canisters on rollers 538.

In some examples, cabinet 500 includes an integrated container 534, which is coupled to a powered tumbler, e.g., at both ends. The integrated container is configured to allow UV light into its interior, e.g., through apertures therein and/or using UV-transparent construction materials. The integrated container is configured to be selectively opened, e.g., using a door thereof, permitting a user to insert and remove objects (e.g., gaming chips), in some examples without removing the container from the cabinet. The container tumbler is powered by motor 536 or the like, which rotates the tumbler using belts, rollers, gears, chains, and/or any suitable combination thereof. The container tumbler rotates the integrated container 534, mechanically agitating chips, tokens, and/or the like contained within, thereby exposing each item to UV light.

### F. Fourth Illustrative UV Sanitizing Cabinet

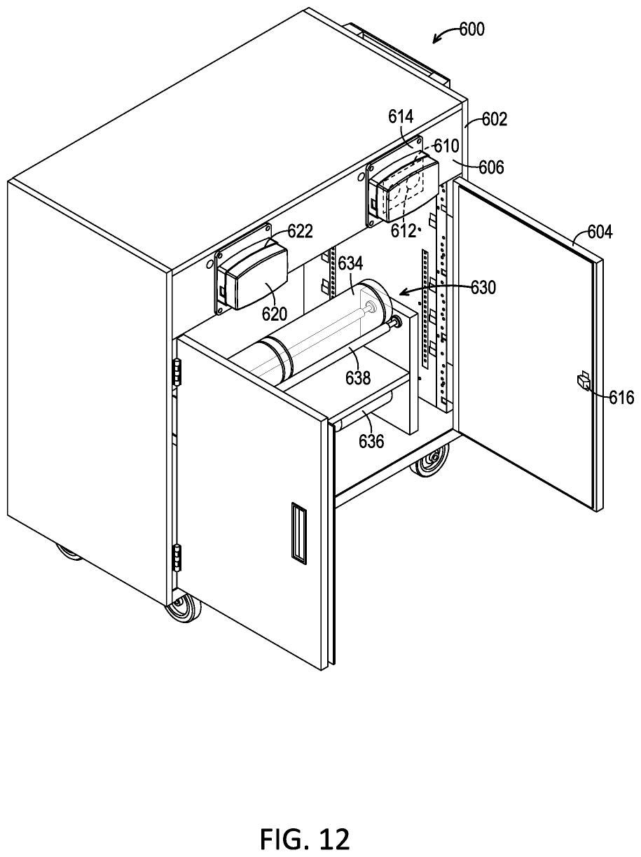

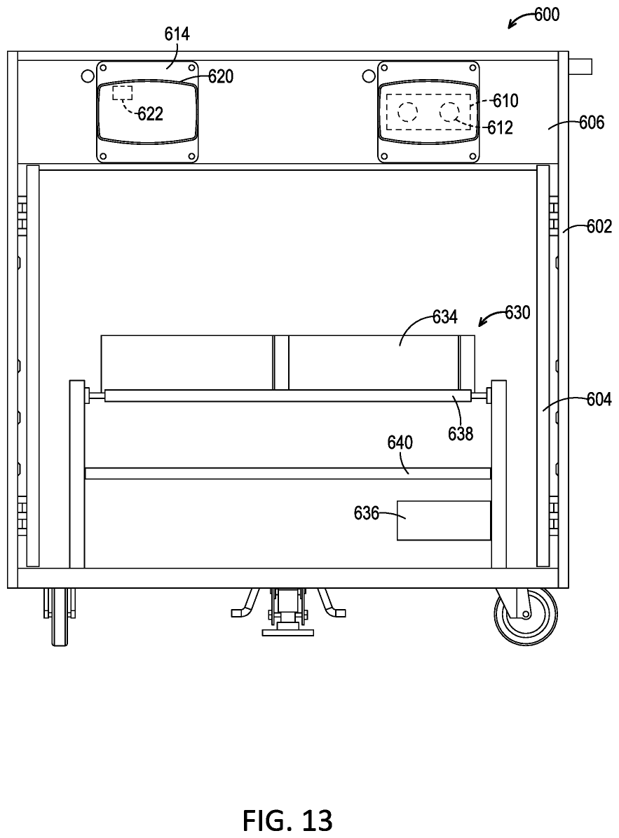

A fourth example of a UV sanitizing cabinet is depicted in FIGS. 12-13 and described below. Cabinet 600 is substantially similar to cabinets 100, 200, and 500, described above.

Cabinet 600 includes a generally cuboidal enclosure 602 having a pair of access doors 604 coupled to a front face of the cabinet by respective hinges (e.g., at outboard sides of the doors). Door 604 is shorter (i.e., lower in height) than the overall face of the enclosure, and a panel 606 is disposed between the door and a top of the enclosure. Two UV light assemblies 610 are each coupled to the cabinet at panel 606, such that respective UV light elements 612 (or sets of elements 612) of each assembly protrude (e.g., through panel 606) into an upper portion of the interior of the cabinet. Each light assembly is secured in place by a respective mounting plate 614, and a respective human-machine interface (HMI) 620 of each light assembly is disposed on an outer surface of each mounting plate, accessible to a user. In some examples, a locking mechanism 616 is disposed on an interior surface of one or more of door(s) 604. Locking mechanism 616 may comprise any suitable electrically or mechanically actuatable locking system(s), such as electromagnets, latches, tumblers, and/or the like. In some examples, cabinet 600 includes a divider disposed between the UV light assemblies, such that each assembly individually illuminates only a portion of the interior of the cabinet. The divider may comprise UVC-blocking (i.e., UV-opaque) material such as steel.

Each light assembly 610 comprises a respective electronics module 622 coupled to one or more of the UV light elements 612 and configured to control the light element(s) to illuminate and/or irradiate the interior of the cabinet. Electronics module(s) 622 may include any suitable electronic controller configured to monitor, control, and/or display information regarding the UV light elements and the overall UV light assembly generally. For example, electronics module 622 may include an electronic controller coupled to one or more sensors (e.g., a door open/closed sensor), and/or HMI 620. In some examples, electronics module 622 includes a wireless and/or wired transmitter, receiver, and/or transceiver for remote monitoring. The electronic controller may be configured to control operation of the UV cabinet in any suitable manner, such as described above with respect to cabinet 100.

Cabinet 600 includes one or more gaming chip sanitizing devices 630 disposed in the interior of the cabinet. In the example depicted in FIGS. 12 and 13, chip sanitizing device 630 includes a plurality of rollers 638 which function to rotate canister(s) 634, the canisters being configured to contain a plurality of chips, coins, tokens, and/or the like (e.g., as described above with respect to canister sanitizer 400). Rotating the canister(s) provides more even UV exposure, and may mechanically agitate chips, tokens, and/or the like contained within each canister, thereby exposing each item to UV light. Canisters 634 comprise material at least partially transparent to UV light, and/or are otherwise configured to allow UV light to illuminate canister contents (e.g., by including a plurality of apertures). Canisters 634 may be substantially similar or identical to canisters 300, described above. Additionally or alternatively, other types of canisters or containers can be rotated within the illuminated internal space of the cabinet.

Chip sanitizing device 630 includes one or more motors 636, in this case disposed beneath a shelf 640 of enclosure 602. In some examples, the shelf comprises UV-blocking material and may shield the motor from UV radiation. Motor 636 causes rotation of a canister drive feature of the chip sanitizing device, e.g., using belts, rollers, gears, chains, and/or any suitable combination thereof. Each chip sanitizing device 630 includes one or more rollers 638 driven by motor 636. In the example depicted in FIG. 12, these rollers are disposed substantially parallel to a front surface of the cabinet enclosure, although other suitable orientations may be utilized, i.e., transverse to the front surface. Power may be transmitted from motor 636 to rollers 638 using any suitable transmission method, e.g., using a direct-drive, gearing, a gearbox, a belt or chain, and/or the like. Cabinet 600 is configured to receive one or more canisters on rollers 638.

In some examples, cabinet 600 is configured to receive and rotate two canisters 634 simultaneously, e.g., disposed adjacent to each other and parallel to a front face of the cabinet housing. In some examples, both canisters rest on a same set of rollers. In some examples, each canister rests on a different set of rollers, and/or is otherwise individually rotated.

In some examples, cabinet 600 includes one or more integrated containers 634, which are respectively coupled to a powered tumbler, e.g., at both ends. Each integrated container is configured to allow UV light into its interior, e.g., through apertures therein and/or using UV-transparent construction materials. Each integrated container is configured to be selectively opened, e.g., using a door thereof, permitting a user to insert and remove objects (e.g., gaming chips), in some examples without removing the container from the cabinet. The container tumbler is powered by motor 636 or the like, which rotates the tumbler using belts, rollers, gears, chains, and/or any suitable combination thereof. The container tumbler rotates the integrated container 634, mechanically agitating chips, tokens, and/or the like contained within, thereby exposing each item to UV light. In some examples, cabinet 600 includes one set of rollers 638, configured to rotate a removable canister, and one integrated container coupled to a tumbler.

Features and mechanisms of the cabinets described herein and shown in the drawings may be combined with features of the other cabinets. For example, the shelf shown in FIG. 1 may be included in the cabinet form factor shown in FIG. 12.

### G. Fifth Illustrative UV Sanitizing Cabinet

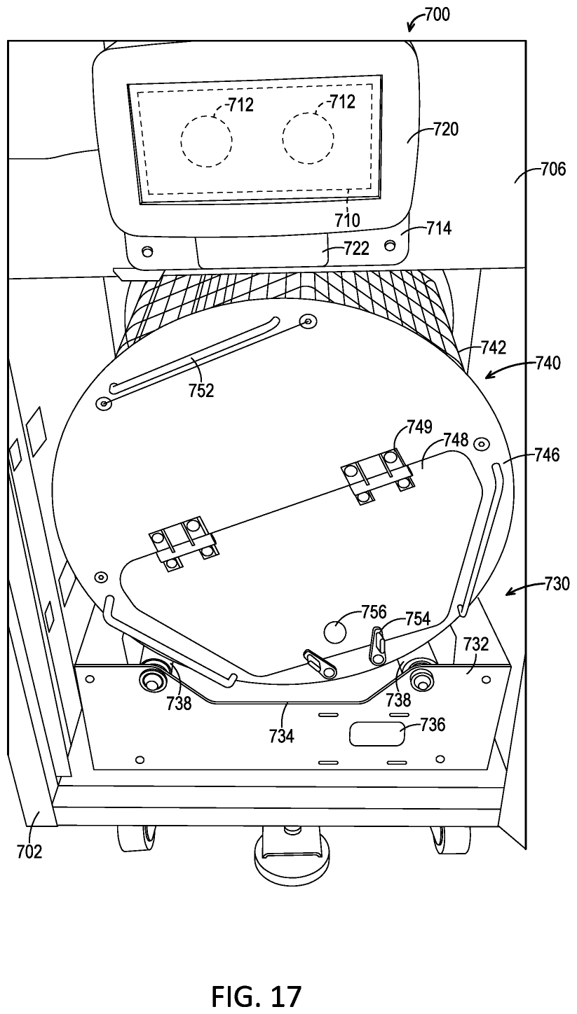

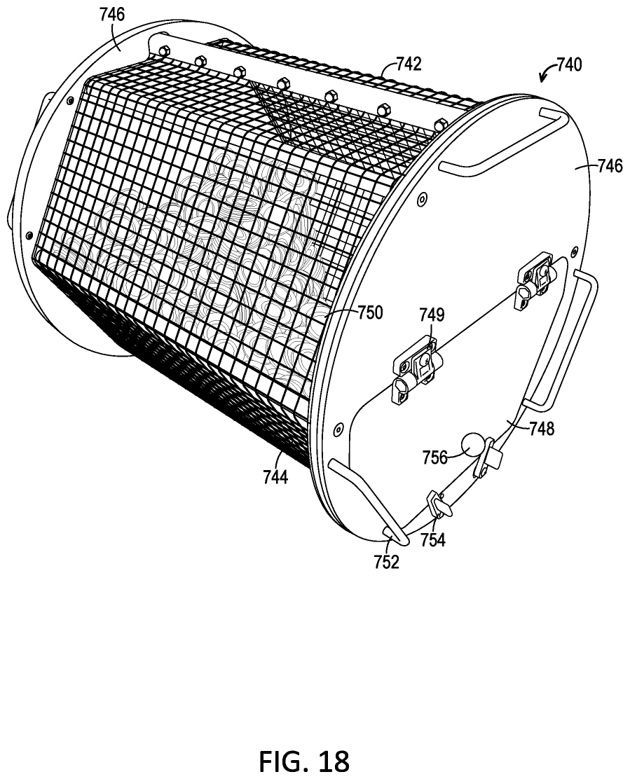

A fifth example of a UV sanitizing cabinet is depicted in FIGS. 17-18 and described below. Cabinet 700 is substantially similar to cabinets 100, 200, 500, and 600, described above.

Cabinet 700 includes a generally cuboidal enclosure 702 having at least one access door (not shown) coupled to a front face by hinges (e.g., side hinges), such as those described above with respect to cabinets 100 and 500. The at least one access door is shorter (i.e., has a smaller height) than the overall face of the enclosure, and a panel 706 is disposed on the front face of the cabinet. A UV light assembly 710 is coupled to the cabinet at panel 706, such that the UV light element(s) 712 protrude (e.g., through panel 706) into the upper portion of the interior of the cabinet. The light assembly is secured in place by a mounting plate 714, and a human-machine interface (HMI) 720 of the light assembly is disposed and accessible on an outer surface of the mounting plate. In some examples, a locking mechanism is disposed on an interior surface of the door. The locking mechanism may comprise any suitable electrically and/or mechanically actuatable locking system, such as electromagnets, latches, tumblers, and/or the like and may be substantially identical to locking mechanisms described and depicted with respect to cabinets described above.

The enclosure and door comprise a rigid structural material that is opaque to UV light, such as steel or plastic. In some examples, interior walls of the cabinet are at least partly reflective, and may comprise aluminum, aluminum paneling, aluminum coatings (e.g., paint), and/or aluminum linings (e.g., foil).

Light assembly 710 includes an electronics module 722 coupled to one or more of the UV light elements 712 (in this case, a pair), configured to illuminate and/or irradiate the interior of the cabinet. Electronics module 722 may include any suitable electronic controller configured to monitor, control, and/or display information regarding the UV light elements and the overall UV light assembly generally. For example, electronics module 722 may include an electronic controller coupled to one or more sensors (e.g., a door open/closed sensor), and/or HMI 720 described above. In some examples, electronics module 722 includes a wireless or wired transmitter, receiver, and/or transceiver for remote monitoring. The electronic controller may be configured to control operation of the UV cabinet in any suitable manner, such as described above with respect to cabinet 100.

Cabinet 700 includes a casino chip sanitizing device 730 disposed in the interior of the cabinet. In the example depicted in FIG. 17, chip sanitizing device 730 includes a plurality of rollers 738 which function to rotate a drum 740. Drum 740 is configured to contain a plurality of gaming chips, tokens, and/or the like (e.g., as described above with respect to canister sanitizer 400). Rotating the drum provides a more even UV exposure for the drum, and may mechanically agitate the contents of the drum, thereby better exposing each item to the UV light.

Drum 740 is depicted in FIG. 18 and includes mesh walls 742 (e.g., at least partially comprising an open mesh), forming a drum interior 744 having a polygonal cross section. Drum 740 includes two disc-shaped end caps 746 coupled to the mesh walls at both ends, thereby forming a substantially cylindrical drum shape. At least one of the end caps 746 includes a door 748 pivotably coupled to the end caps by hinges 749, which is configured to provide access to drum interior 744. Drum 740 is configured to maximize UV exposure to chips 750 contained therein. Mesh walls 742 may comprise openings of any suitable shape, such as square, hexagonal, triangular, and/or the like. In some examples, mesh walls 742 comprise a single piece of mesh screen, bolted together at ends to form a roughly cylindrical mesh tube. A largest dimension of the mesh openings may be configured to be smaller than a diameter of an average casino chip, such that chips do not fall through the mesh openings when rotated. In some examples, mesh walls 742 may comprise a metal mesh covered with any suitable resilient polymer configured to protect chips 750 from damage such as rubber, polyurethane, polyethylene, and/or the like. Drum 740 may have any suitable cross section configured to facilitate chip movement within the drum, such as hexagonal, octagonal, round, and/or the like.

End caps 746 may be configured to facilitate ease of insertion and removal of both drum 740 into cabinet 700 and chips 750 into drum 740. In some examples, drum 740 includes handles 752 disposed near edges of end caps 746 which enable a user to remove the drum from the cabinet. Door 748 may include one or more latches 754 disposed opposite hinges 749, which may prevent the door from opening while the drum is rotating. Door 748 may further include a knob 756 disposed opposite hinges 749, which may facilitate opening the door. In some examples, door 748 may facilitate insertion of chips into drum 740 without removal of the drum from cabinet 700.

Chip sanitizing device 730 includes a drive motor 736, in this case disposed within a housing 732 of the sanitizing device. Housing 732 includes a recess 734 configured to receive drum 740 as the drum rotates. In some examples, housing 732 comprises a UV-blocking material and is configured to shield the motor from UV radiation. Motor 736 causes rotation of a drum drive feature of the chip sanitizing device, e.g., using belts, rollers, gears, chains, and/or any suitable combination thereof. Chip sanitizing device 730 includes one or more (e.g., a pair of parallel) rollers 738 driven by motor 736. In the example of FIG. 17, these rollers are disposed substantially transverse to a front surface of the cabinet enclosure and at a bottom surface of recess 734, although other suitable orientations may be utilized (i.e., parallel to the front surface). Power may be transmitted from motor 736 to rollers 738 using any suitable transmission method, e.g., using a direct-drive, gearing, a gearbox, a belt or chain, and/or the like.

### H. Illustrative Method

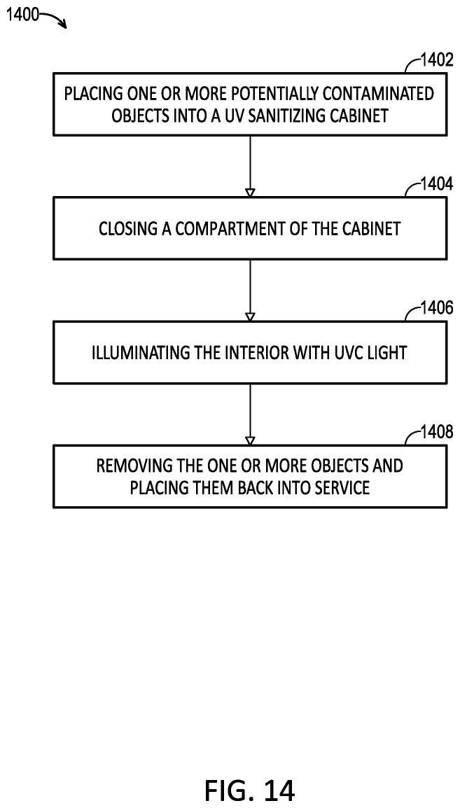

This section describes steps of an illustrative method 1400 for sterilizing small items such as those used in banking and casinos; see FIG. 14. Aspects of UV sanitizing cabinets, canisters, and/or canister sanitizing devices described above may be utilized in the method steps described below. Where appropriate, reference may be made to components and systems that may be used in carrying out each step. These references are for illustration, and are not intended to limit the possible ways of carrying out any particular step of the method.

FIG. 14 is a flowchart illustrating steps performed in an illustrative method, and may not recite the complete process or all steps of the method. Although various steps of method 1400 are described below and depicted in FIG. 14, the steps need not necessarily all be performed, and in some cases may be performed simultaneously or in a different order than the order shown.

Step 1402 of method 1400 includes placing one or more potentially contaminated objects into a compartment of a UV sanitizing cabinet (e.g., cabinet 100), wherein the cabinet includes a rack or container configured to hold the one or more objects. In some examples, the rack includes apertures generally shaped and configured to receive the one or more objects. In some examples, the one or more objects comprise a pneumatic tube carrier or similarly shaped canister. In some examples, step 1402 includes opening the canister clamshell before placing it onto the rack.

Step 1404 of method 1400 includes closing the compartment of the UV cabinet. In some examples, closing the compartment includes closing a door of the UV cabinet. In some examples, closing the compartment includes closing a drawer of the UV cabinet. In some examples, an electronic controller of the cabinet receives information from a sensor that the compartment is shut.

Step 1406 of method 1400 includes illuminating an interior of the cabinet compartment with UV light (e.g., UVC light) using one or more UV-emitting lamps disposed at least partially within the interior. In some examples, step 1406 includes automatically turning the lamp(s) on when the compartment is closed. In some examples, step 1406 is controlled by the electronic controller. In some examples, step 1406 includes illuminating the interior for a selected amount of time. In some examples, the selected amount of time is based on a user-selectable program. In some examples, the selected amount of time is based on a desired or calculated UV dosage. In some examples, illuminating the interior includes using one or more reflectors or reflective surfaces. In some examples, the UV light is left in an “on” condition regardless of compartment, door, and/or drawer position.

In some examples, step 1406 optionally includes interrogating the one or more objects using an RFID reader, and receiving information regarding the one or more objects from RFID tags thereon. UV illumination may be controlled (e.g., automatically), in terms of duration, volume, and/or intensity, based on the received information regarding the one or more objects. For example, the RFID tags may be configured to convey information identifying a specific canister and/or type of canister, and an electronic controller may be configured to select a dosage based on the conveyed information. For example, a canister known to comprise UV-transparent material may be irradiated for a shorter time than a canister comprising a UV-opaque material with UV-admitting apertures.

Step 1408 of method 1400 includes removing the one or more objects from the cabinet and placing them back into service. In some examples, step 1408 includes opening the compartment of the UV cabinet. In some examples, step 1408 includes opening a door. In some examples, step 1408 includes opening a drawer. In some examples, opening the compartment causes an automatic shutoff or disabling of the UV lamps.

### I. Illustrative Data Processing System

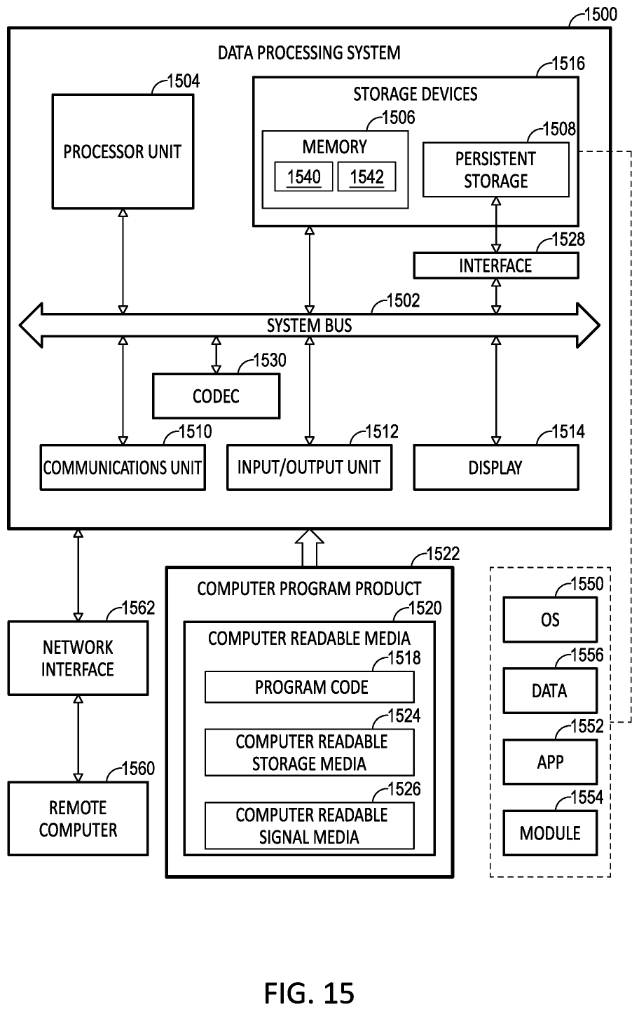

As shown in FIG. 15, this example describes a data processing system 1500 (also referred to as a computer, computing system, and/or computer system) in accordance with aspects of the present disclosure. In this example, data processing system 1500 is an illustrative data processing system suitable for implementing aspects of the UV sanitizing cabinets described above. More specifically, in some examples, devices that are embodiments of data processing systems (e.g., smartphones, tablets, personal computers) may determine ideal UV dosages based on information inputted by a user or received via an RFID system. In some examples, data processing systems may synthesize information about likely microbial strains contaminating an object with UV dosages required to reduce microbe count by a satisfactory amount. For example, in times of pandemic, UV dosages delivered by the UV system may be higher than in times with less disease. In some examples, data processing systems may track different items received within a sanitizing cabinet, as described above, and alert a user when each item has received a specified UV dose.

In this illustrative example, data processing system 1500 includes a system bus 1502 (also referred to as communications framework). System bus 1502 may provide communications between a processor unit 1504 (also referred to as a processor or processors), a memory 1506, a persistent storage 1508, a communications unit 1510, an input/output (I/O) unit 1512, a codec 1530, and/or a display 1514. Memory 1506, persistent storage 1508, communications unit 1510, input/output (I/O) unit 1512, display 1514, and codec 1530 are examples of resources that may be accessible by processor unit 1504 via system bus 1502.

Processor unit 1504 serves to run instructions that may be loaded into memory 1506. Processor unit 1504 may comprise a number of processors, a multi-processor core, and/or a particular type of processor or processors (e.g., a central processing unit (CPU), graphics processing unit (GPU), etc.), depending on the particular implementation. Further, processor unit 1504 may be implemented using a number of heterogeneous processor systems in which a main processor is present with secondary processors on a single chip. As another illustrative example, processor unit 1504 may be a symmetric multi-processor system containing multiple processors of the same type.

Memory 1506 and persistent storage 1508 are examples of storage devices 1516. A storage device may include any suitable hardware capable of storing information (e.g., digital information), such as data, program code in functional form, and/or other suitable information, either on a temporary basis or a permanent basis.

Storage devices 1516 also may be referred to as computer-readable storage devices or computer-readable media. Memory 1506 may include a volatile storage memory 1540 and a non-volatile memory 1542. In some examples, a basic input/output system (BIOS), containing the basic routines to transfer information between elements within the data processing system 1500, such as during start-up, may be stored in non-volatile memory 1542. Persistent storage 1508 may take various forms, depending on the particular implementation.

Persistent storage 1508 may contain one or more components or devices. For example, persistent storage 1508 may include one or more devices such as a magnetic disk drive (also referred to as a hard disk drive or HDD), solid state disk (SSD), floppy disk drive, tape drive, Jaz drive, Zip drive, flash memory card, memory stick, and/or the like, or any combination of these. One or more of these devices may be removable and/or portable, e.g., a removable hard drive. Persistent storage 1508 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 1508 to system bus 1502, a removable or non-removable interface is typically used, such as interface 1528.

Input/output (I/O) unit 1512 allows for input and output of data with other devices that may be connected to data processing system 1500 (i.e., input devices and output devices). For example, an input device may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 1504 through system bus 1502 via interface port(s). Suitable interface port(s) may include, for example, a serial port, a parallel port, a game port, and/or a universal serial bus (USB).

One or more output devices may use some of the same types of ports, and in some cases the same actual ports, as the input device(s). For example, a USB port may be used to provide input to data processing system 1500 and to output information from data processing system 1500 to an output device. One or more output adapters may be provided for certain output devices (e.g., monitors, speakers, and printers, among others) which require special adapters. Suitable output adapters may include, e.g. video and sound cards that provide a means of connection between the output device and system bus 1502. Other devices and/or systems of devices may provide both input and output capabilities, such as remote computer(s) 1560. Display 1514 may include any suitable human-machine interface or other mechanism configured to display information to a user, e.g., a CRT, LED, or LCD monitor or screen, etc.

Communications unit 1510 refers to any suitable hardware and/or software employed to provide for communications with other data processing systems or devices. While communication unit 1510 is shown inside data processing system 1500, it may in some examples be at least partially external to data processing system 1500. Communications unit 1510 may include internal and external technologies, e.g., modems (including regular telephone grade modems, cable modems, and DSL modems), ISDN adapters, and/or wired and wireless Ethernet cards, hubs, routers, etc. Data processing system 1500 may operate in a networked environment, using logical connections to one or more remote computers 1560. A remote computer(s) 1560 may include a personal computer (PC), a server, a router, a network PC, a workstation, a microprocessor-based appliance, a peer device, a smart phone, a tablet, another network note, and/or the like. Remote computer(s) 1560 typically include many of the elements described relative to data processing system 1500. Remote computer(s) 1560 may be logically connected to data processing system 1500 through a network interface 1562 which is connected to data processing system 1500 via communications unit 1510. Network interface 1562 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 1530 may include an encoder, a decoder, or both, comprising hardware, software, or a combination of hardware and software. Codec 1530 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 1530 is depicted as a separate component, codec 1530 may be contained or implemented in memory, e.g., non-volatile memory 1542.

Non-volatile memory 1542 may include read only memory (ROM), programmable ROM (PROM), electrically programmable ROM (EPROM), electrically erasable programmable ROM (EEPROM), flash memory, and/or the like, or any combination of these. Volatile memory 1540 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 1516, which are in communication with processor unit 1504 through system bus 1502. In these illustrative examples, the instructions are in a functional form in persistent storage 1508. These instructions may be loaded into memory 1506 for execution by processor unit 1504. Processes of one or more embodiments of the present disclosure may be performed by processor unit 1504 using computer-implemented instructions, which may be located in a memory, such as memory 1506.

These instructions are referred to as program instructions, program code, computer usable program code, or computer-readable program code executed by a processor in processor unit 1504. The program code in the different embodiments may be embodied on different physical or computer-readable storage media, such as memory 1506 or persistent storage 1508. Program code 1518 may be located in a functional form on computer-readable media 1520 that is selectively removable and may be loaded onto or transferred to data processing system 1500 for execution by processor unit 1504. Program code 1518 and computer-readable media 1520 form computer program product 1522 in these examples. In one example, computer-readable media 1520 may comprise computer-readable storage media 1524 or computer-readable signal media 1526.

Computer-readable storage media 1524 may include, for example, an optical or magnetic disk that is inserted or placed into a drive or other device that is part of persistent storage 1508 for transfer onto a storage device, such as a hard drive, that is part of persistent storage 1508. Computer-readable storage media 1524 also may take the form of a persistent storage, such as a hard drive, a thumb drive, or a flash memory, that is connected to data processing system 1500. In some instances, computer-readable storage media 1524 may not be removable from data processing system 1500.

In these examples, computer-readable storage media 1524 is a non-transitory, physical or tangible storage device used to store program code 1518 rather than a medium that propagates or transmits program code 1518. Computer-readable storage media 1524 is also referred to as a computer-readable tangible storage device or a computer-readable physical storage device. In other words, computer-readable storage media 1524 is media that can be touched by a person.

Alternatively, program code 1518 may be transferred to data processing system 1500, e.g., remotely over a network, using computer-readable signal media 1526. Computer-readable signal media 1526 may be, for example, a propagated data signal containing program code 1518. For example, computer-readable signal media 1526 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 1518 may be downloaded over a network to persistent storage 1508 from another device or data processing system through computer-readable signal media 1526 for use within data processing system 1500. For instance, program code stored in a computer-readable storage medium in a server data processing system may be downloaded over a network from the server to data processing system 1500. The computer providing program code 1518 may be a server computer, a client computer, or some other device capable of storing and transmitting program code 1518.

In some examples, program code 1518 may comprise an operating system (OS) 1550. Operating system 1550, which may be stored on persistent storage 1508, controls and allocates resources of data processing system 1500. One or more applications 1552 take advantage of the operating system's management of resources via program modules 1554, and program data 1556 stored on storage devices 1516. OS 1550 may include any suitable software system configured to manage and expose hardware resources of computer 1500 for sharing and use by applications 1552. In some examples, OS 1550 provides application programming interfaces (APIs) that facilitate connection of different type of hardware and/or provide applications 1552 access to hardware and OS services. In some examples, certain applications 1552 may provide further services for use by other applications 1552, e.g., as is the case with so-called “middleware.” Aspects of present disclosure may be implemented with respect to various operating systems or combinations of operating systems.

The different components illustrated for data processing system 1500 are not meant to provide architectural limitations to the manner in which different embodiments may be implemented. One or more embodiments of the present disclosure may be implemented in a data processing system that includes fewer components or includes components in addition to and/or in place of those illustrated for computer 1500. Other components shown in FIG. 15 can be varied from the examples depicted. Different embodiments may be implemented using any hardware device or system capable of running program code. As one example, data processing system 1500 may include organic components integrated with inorganic components and/or may be comprised entirely of organic components (excluding a human being). For example, a storage device may be comprised of an organic semiconductor.

In some examples, processor unit 1504 may take the form of a hardware unit having hardware circuits that are specifically manufactured or configured for a particular use, or to produce a particular outcome or progress. This type of hardware may perform operations without needing program code 1518 to be loaded into a memory from a storage device to be configured to perform the operations. For example, processor unit 1504 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 1518) may be implemented as hardware, e.g., by specifying an FPGA configuration using a hardware description language (HDL) and then using a resulting binary file to (re)configure the FPGA.

In another example, data processing system 1500 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 1504 may be implemented using a combination of processors found in computers and hardware units. Processor unit 1504 may have a number of hardware units and a number of processors that are configured to run program code 1518. With this depicted example, some of the processes may be implemented in the number of hardware units, while other processes may be implemented in the number of processors.

In another example, system bus 1502 may comprise one or more buses, such as a system bus or an input/output bus. Of course, the bus system may be implemented using any suitable type of architecture that provides for a transfer of data between different components or devices attached to the bus system. System bus 1502 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI)).

Additionally, communications unit 1510 may include a number of devices that transmit data, receive data, or both transmit and receive data. Communications unit 1510 may be, for example, a modem or a network adapter, two network adapters, or some combination thereof. Further, a memory may be, for example, memory 1506, or a cache, such as that found in an interface and memory controller hub that may be present in system bus 1502.

### J. Illustrative Distributed Data Processing System

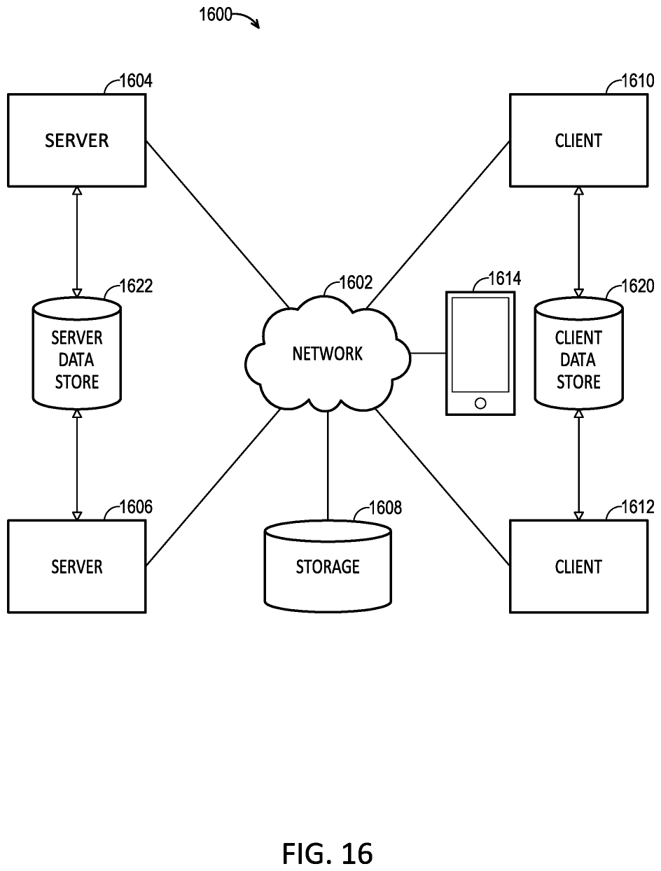

As shown in FIG. 16, this example describes a general network data processing system 1600, interchangeably termed a computer network, a network system, a distributed data processing system, or a distributed network, aspects of which may be included in one or more illustrative embodiments of UV sanitizing cabinets described above. For example, a human-machine interface included in a UV sanitizing cabinet may be embodied as a smartphone or tablet, and may communicate with an electronics module of the cabinet using a networked system. In some examples, UV sanitizing cabinets may be operated by users (e.g., bank tellers, casino dealers) in large organizations, such as corporate or other business entities. In some examples, administrators may set specified UV doses for all sanitizing cabinets used in an organization. In some examples, administrators may track sanitizing histories for all canisters in an organization using RFID systems, as described above and below.

It should be appreciated that FIG. 16 is provided as an illustration of one implementation and is not intended to imply any limitation with regard to environments in which different embodiments may be implemented. Many modifications to the depicted environment may be made.

Network system 1600 is a network of devices (e.g., computers), each of which may be an example of data processing system 1500, and other components. Network data processing system 1600 may include network 1602, which is a medium configured to provide communications links between various devices and computers connected within network data processing system 1600. Network 1602 may include connections such as wired or wireless communication links, fiber optic cables, and/or any other suitable medium for transmitting and/or communicating data between network devices, or any combination thereof.

In the depicted example, a first network device 1604 and a second network device 1606 connect to network 1602, as do one or more computer-readable memories or storage devices 1608. Network devices 1604 and 1606 are each examples of data processing system 1500, described above. In the depicted example, devices 1604 and 1606 are shown as server computers, which are in communication with one or more server data store(s) 1622 that may be employed to store information local to server computers 1604 and 1606, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 1610 and 1612 and/or a client smart device 1614, may connect to network 1602. Each of these devices is an example of data processing system 1500, described above regarding FIG. 15. Client electronic devices 1610, 1612, and 1614 may include, for example, one or more personal computers, network computers, and/or mobile computing devices such as personal digital assistants (PDAs), smart phones, handheld gaming devices, wearable devices, and/or tablet computers, and the like. In the depicted example, server 1604 provides information, such as boot files, operating system images, and applications to one or more of client electronic devices 1610, 1612, and 1614. Client electronic devices 1610, 1612, and 1614 may be referred to as “clients” in the context of their relationship to a server such as server computer 1604. Client devices may be in communication with one or more client data store(s) 1620, which may be employed to store information local to the clients (e,g., cookie(s) and/or associated contextual information). Network data processing system 1600 may include more or fewer servers and/or clients (or no servers or clients), as well as other devices not shown.

In some examples, first client electric device 1610 may transfer an encoded file to server 1604. Server 1604 can store the file, decode the file, and/or transmit the file to second client electric device 1612. In some examples, first client electric device 1610 may transfer an uncompressed file to server 1604 and server 1604 may compress the file. In some examples, server 1604 may encode text, audio, and/or video information, and transmit the information via network 1602 to one or more clients.

Client smart device 1614 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may include features of other known electronic devices, such as a media player, personal digital assistant, digital camera, video camera, and/or global positioning system. Smart devices (e.g., smartphones) may be capable of connecting with other smart devices, computers, or electronic devices wirelessly, such as through near field communications (NFC), BLUETOOTH®, WiFi, or mobile broadband networks. Wireless connectively may be established among smart devices, smartphones, computers, and/or other devices to form a mobile network where information can be exchanged.

Data and program code located in system 1600 may be stored in or on a computer-readable storage medium, such as network-connected storage device 1608 and/or a persistent storage 1508 of one of the network computers, as described above, and may be downloaded to a data processing system or other device for use. For example, program code may be stored on a computer-readable storage medium on server computer 1604 and downloaded to client 1610 over network 1602, for use on client 1610. In some examples, client data store 1620 and server data store 1622 reside on one or more storage devices 1608 and/or 1508.

Network data processing system 1600 may be implemented as one or more of different types of networks. For example, system 1600 may include an intranet, a local area network (LAN), a wide area network (WAN), or a personal area network (PAN). In some examples, network data processing system 1600 includes the Internet, with network 1602 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 1602 may be referred to as a “cloud.” In those examples, each server 1604 may be referred to as a cloud computing node, and client electronic devices may be referred to as cloud consumers, or the like. FIG. 16 is intended as an example, and not as an architectural limitation for any illustrative embodiments.

### K. Illustrative Radio-Frequency Identification Systems

In general, radio-frequency identification (RFID) refers to wireless and typically non-contact use of radio-frequency waves to transfer data. An RFID system includes at least one RFID tag readable by at least one RFID reader. The tag typically includes an integrated circuit (AKA a chip) coupled to a tag antenna. The integrated circuit is configured to modulate a signal such that the signal contains predetermined information including a unique identifier. The tag antenna is configured to transmit the modulated signal (e.g., when interrogated by the reader). The reader is configured to read the tag by receiving the modulated signal using a reader antenna coupled to, or integral with, the reader. Reading the tag in this manner allows the reader to identify the tag based on the data (i.e., the unique identifier) embodied in the modulated signal.

In some examples, UV sanitizing cabinets and canisters described herein may utilized RFID systems to track cabinet contents and standardize UVC dosages. UV sanitizing cabinets may include RFID readers disposed above one or more doors of the cabinets. Canisters designed for use with the UV sanitizing cabinets may each include an RFID tag on an interior or exterior surface. In some examples, an electronics module coupled to an RFID reader may count a number of canisters disposed within the sanitizing cabinet. This value may be utilized to determine an ideal UV dosage for canisters disposed within the cabinet. In some examples, a server of a distributed data processing system may track a sanitization history for specific canisters based on a number of times an RFID tag has been read by an RFID reader. In some examples, specific canisters or other objects may be assigned specific sanitization programs. The UV sanitizing cabinet may apply a specific UV dose to a specific canister based on information received from the RFID tag.

Specific embodiments of an RFID system may be characterized by the frequency of the signal used, on whether the signal to be modulated is generated within the tag or within the reader, on the specific type of electromagnetic coupling between the reader and the tag, and/or on any other suitable factor(s).

For example, an RFID system may include one or more passive tags, semi-passive tags, and/or active tags. A passive RFID tag includes no power source and is configured to receive energy (e.g., through inductive coupling) in the form of radio-frequency waves transmitted by the RFID reader. The received radio-frequency wave is modulated by the circuit of the RFID tag (e.g., via load modulation) and transmitted by the tag's antenna to the reader. In other words, the reader interrogates the passive RFID tag by transmitting a radio-frequency signal to the tag, and then receives a modulated version of the radio-frequency signal.

Like a passive RFID tag, a semi-passive RFID tag is configured to receive a radio-frequency signal from the reader, modulate the signal, and transmit the modulated version of the signal back to the reader. Unlike a passive tag, however, the semi-passive RFID tag does include a power source (e.g., a battery). The power source of the semi-passive RFID tag may provide power to the circuit of the tag, and/or to additional sensors or circuits included in or accompanying the tag. However, the power source does not generate the signal to be transmitted to the reader.

In contrast, an active RFID tag includes a power supply, and uses the power supply to generate a modulated signal to be sent to the reader. In other words, the signal read by the reader originates in the RFID tag, rather than in the reader (as is the case when a passive or semi-passive RFID tag is used). In some examples, an active RFID tag includes additional sensors or circuitry powered by the tag power source.

Some examples of active RFID tags are configured to transmit a modulated signal at a predetermined interval (e.g., every several seconds). An active RFID tag operating in this mode may be referred to as a beacon. Alternatively, or additionally, an active RFID tag may be configured to transmit the modulated signal to the reader in response to receiving a wake-up signal from the reader. A tag operating in this mode may be referred to as an active transponder tag.

RFID systems may be further characterized based on whether they use low-frequency, high-frequency, or ultra-high-frequency radio waves. In an RFID context, the low-frequency (LF) range typically comprises waves having frequencies in the range of 30 kHz to 300 kHz. In some geographic locations, and/or for certain applications, low-frequency RFID systems are required by local laws or rules to operate within a narrower range, such as 125 kHz to 134 kHz, to avoid interference with other signals. Tags of a low-frequency RFID system are typically passive tags configured to couple to an RFID reader via magnetic coupling. Radio waves in the low-frequency range generally propagate through water and/or certain other liquids with little to no absorption. For at least this reason, LF RFID systems may be suitable for applications where the tag or the reader is disposed adjacent a liquid. Typically, LF tags are readable by LF readers disposed at a maximum distance of several centimeters to tens of centimeters, depending on environmental conditions.

High-frequency (HF) RFID systems typically use radio waves having frequencies in the range of 3 MHz to 30 MHz. An HF tag is typically a passive tag powered by and read by an HF reader using inductive coupling. HF RFID systems may have a read range of several centimeters up to approximately a meter. Compared to LF signals, HF signals are more readily absorbed by water and metal. Accordingly, HF systems tend to work poorly in cases where the signal may need to pass through water (or water-based liquids), or through thick layers of metal.

One example of an HF RFID system is a near-field communication (NFC) system, which is a global communication standard operating at 13.56 MHz. In some examples, an NFC device is configured to act as both a reader and a tag (e.g., in a peer-to-peer communication NFC system). Such an NFC device is typically also capable of reading a passive NFC tag, and is in some cases also capable of reading a passive HF RFID tag compliant with suitable communication standards.

Ultra-high frequency (UHF) RFID systems generally operate in the 300 MHz to 3 GHz range. Due to commonly used communication standards and/or regulations, many examples of UHF systems operate either in the range of 860 to 960 MHz, at 433 MHz, or at 2.45 GHz. Some UHF RFID systems are active, and some are passive. Passive UHF RFID tags typically couple to UHF readers electromagnetically via backscatter modulation, rather than the inductive coupling used by typical LF and HF passive systems. One benefit of UHF tags is their range. A passive UHF RFID tag may be readable by a UHF reader at a maximum distance of tens of meters. The read range of an active UHF tag depends on the power of the signal transmitted by the tag, among other factors. UHF waves interact readily with liquid and metal (e.g., by reflection, absorption, and/or refraction), and UHF systems therefore typically perform poorly in the presence of these substances unless mitigating techniques or devices are used.

### L. Illustrative Combinations and Additional Examples

This section describes additional aspects and features of UV sanitizing cabinets, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A0. A sanitizing cabinet comprising:

a cabinet housing including one or more walls defining an interior space;

an ultraviolet (UV) light-emitting device coupled to the cabinet housing such that the UV light-emitting device is configured to emit UV light into the interior space; and

a receiving apparatus configured to securely hold a canister within the interior space in a position exposed to the UV light.

A1. The cabinet of A0, further comprising the canister, wherein the canister is generally cylindrical and is configured to selectively hold one or more small objects.

A2. The cabinet of A1, wherein the canister comprises a UV-transmissive material.

A3. The cabinet of A2, wherein the material is fluorinated ethylene propylene.

A4. The cabinet of A1, wherein the canister comprises a material opaque to UV light and having a plurality of UV light-admitting apertures formed therein.

A5. The cabinet of any one of paragraphs A0 through A4, wherein the receiving apparatus comprises a shelf disposed within the interior space, the shelf comprising a grid defining a plurality of openings each configured to support the canister.

A6. The cabinet of any one of paragraphs A0 through A5, wherein the cabinet housing further comprises one or more UV-reflective surfaces disposed within the interior space.

A7. The cabinet of any one of paragraphs A0 through A6, further comprising:

a tiltable container having a front panel pivotably coupled at a bottom edge to the cabinet housing, such that pivoting the front panel about the bottom edge tilts the tiltable container between a first position, wherein the tiltable container is contained within the interior space, and a second position, wherein the tiltable container is at least partially tilted out of the interior space and an open top of the tiltable container is accessible by a user.

A8. The cabinet of any one of paragraphs A0 through A7, further comprising a drawer disposed in an upper portion of the cabinet above the light-emitting device.

A9. The cabinet of A8, wherein a portion of the drawer is configured to allow ultraviolet radiation therethrough.

A10. The cabinet of A9, wherein a bottom of the drawer comprises a material opaque to ultraviolet radiation and has one or more openings configured to allow transmission of ultraviolet radiation.

A11. The cabinet of any one of paragraphs A0 through A10, wherein the receiving apparatus comprises a powered roller system configured to rotate the canister during exposure to the UV light.

A12. The cabinet of any one of paragraphs A0 through A11, further comprising an RFID reader configured to read an RFID tag of an object disposed in the interior space, and wherein a controller of the light-emitting device is in electronic communication with the RFID reader and configured to automatically emit a selected dosage of ultraviolet radiation based on information from the RFID tag.

B0. A sanitizing system comprising:

a cabinet including a cabinet housing defining a compartment;

an ultraviolet (UV) radiation-producing device coupled to the cabinet;

an electronic controller coupled to the UV radiation-producing device and configured to control the UV radiation-producing device to selectively emit ultraviolet radiation into the compartment;

a canister having at least one canister wall that is transmissive with respect to ultraviolet radiation; and

a canister support structure disposed within the compartment and configured to receive the canister, such that contents of the canister are exposed to the ultraviolet radiation from the UV radiation-producing device through the canister wall.

B1. The sanitizing system of B0, wherein the support structure comprises a container pivotably coupled to the cabinet housing and selectively tiltable out of the compartment.

B2. The sanitizing system of B0 or B1, wherein the support structure includes at least one motorized roller configured to rotate the canister during UV exposure.

B3. The sanitizing system of any one of paragraphs B0 through B2, further comprising a slidable drawer disposed in the cabinet housing above the compartment.

B4. The sanitizing system of B3, wherein a floor of the slidable drawer is configured to allow transmission of ultraviolet radiation emitted by the UV radiation-producing device into the slidable drawer.

B5. The sanitizing system of any one of paragraphs B0 through B4, wherein the electronic controller is further configured to automatically recognize a canister held by the support structure, to automatically determine a dosage based on data related to the recognized canister, and to automatically control the radiation-producing device to emit ultraviolet radiation based on the determined dosage.

B6. The sanitizing system of B5, wherein automatically determining the dosage includes automatically determining at least one of a radiation intensity and an exposure time.

B7. The sanitizing system of B5, further comprising a transmitter coupled to the electronic controller and configured to communicate information about the recognized canister to a server.

B8. The sanitizing system of B7, wherein the transmitter comprises a transceiver and is further configured to receive dosage data from the server, and wherein the data related to the recognized canister includes the dosage data.

C0. A method for sanitizing small objects such as gaming (e.g., poker) chips, the method comprising:

opening an access door of a cabinet having an ultraviolet (UV) lamp housed therein;

placing a canister into a canister receiving structure of the cabinet through the access door, wherein the canister contains an object to be sanitized;

closing the access door and, using the UV lamp, irradiating the object in the canister with UV light for a selected duration, wherein the object is exposed to the UV light through a wall of the canister;

increasing exposure of the object to the UV light by automatically reorienting the canister during the selected duration.

C1. The method of C0, wherein the canister receiving structure comprises a powered roller assembly configured to automatically rotate the canister about a long axis thereof.

C2. The method of C0 or C1, wherein the wall of the canister comprises a material having a transmittance of at least 90% with respect to UV light, e.g., FEP.

### Advantages, Features, and Benefits

The different embodiments and examples of the UV sanitizing cabinets described herein provide several advantages over known solutions for sanitizing banking equipment. For example, illustrative embodiments and examples described herein allow a bank teller to ensure objects used by customers and tellers are sanitized periodically.

Additionally, and among other benefits, illustrative embodiments and examples described herein allow a bank teller to control their own equipment sterilization.

Additionally, and among other benefits, illustrative embodiments and examples described herein allow a user to ensure sanitization by utilizing preprogrammed UV light regimes (duration, volume—e.g., number of lamps activated, intensity, etc.).

Additionally, and among other benefits, illustrative embodiments and examples described herein are easily accessible and usable by a teller, allowing real-time sanitization of equipment and quick return to service, e.g., because the cabinets are located adjacent the user while the user is performing normal duties.

Additionally, and among other benefits, illustrative embodiments and examples described herein allow the automatic identification of one or more objects within the cabinet (e.g., using RFID), resulting for example in the automatic use of corresponding sanitization programs (e.g., custom programs) based on the identified object(s).

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed is:

1. A sanitizing cabinet comprising:
a cabinet housing including one or more walls defining an interior space;
an ultraviolet (UV) light-emitting device coupled to the cabinet housing such that the UV light-emitting device is configured to emit UV light into the interior space; and
a receiving apparatus configured to securely hold a canister within the interior space in a position exposed to the UV light.

2. The cabinet of claim 1, further comprising the canister, wherein the canister is generally cylindrical and is configured to selectively hold one or more small objects.

3. The cabinet of claim 2, wherein the canister comprises a UV-transmissive material.

4. The cabinet of claim 3, wherein the material is fluorinated ethylene propylene.

5. The cabinet of claim 2, wherein the canister comprises a material opaque to UV light and having a plurality of UV light-admitting apertures formed therein.

6. The cabinet of claim 1, wherein the receiving apparatus comprises a shelf disposed within the interior space, the shelf comprising a grid defining a plurality of openings each configured to support the canister.

7. The cabinet of claim 1, wherein the cabinet housing further comprises one or more UV-reflective surfaces disposed within the interior space.

8. The cabinet of claim 1, further comprising:
a tiltable container having a front panel pivotably coupled at a bottom edge to the cabinet housing, such that pivoting the front panel about the bottom edge tilts the tiltable container between a first position, wherein the tiltable container is contained within the interior space, and a second position, wherein the tiltable container is at least partially tilted out of the interior space and an open top of the tiltable container is accessible by a user.

9. The cabinet of claim 1, further comprising a drawer disposed in an upper portion of the cabinet above the light-emitting device.

10. The cabinet of claim 9, wherein a portion of the drawer is configured to allow ultraviolet radiation therethrough.

11. The cabinet of claim 10, wherein a bottom of the drawer comprises a material opaque to ultraviolet radiation and has one or more openings configured to allow transmission of ultraviolet radiation.

12. The cabinet of claim 1, wherein the receiving apparatus comprises a powered roller system configured to rotate the canister during exposure to the UV light.

13. A sanitizing system comprising:
a cabinet including a cabinet housing defining a compartment;
an ultraviolet (UV) radiation-producing device coupled to the cabinet;
an electronic controller coupled to the UV radiation-producing device and configured to control the UV radiation-producing device to selectively emit ultraviolet radiation into the compartment;
a canister having at least one canister wall that is transmissive with respect to ultraviolet radiation; and
a canister support structure disposed within the compartment and configured to receive the canister, such that contents of the canister are exposed to the ultraviolet radiation from the UV radiation-producing device through the canister wall.

14. The sanitizing system of claim 13, wherein the support structure comprises a container pivotably coupled to the cabinet housing and selectively tiltable out of the compartment.

15. The sanitizing system of claim 13, wherein the support structure includes at least one motorized roller configured to rotate the canister during UV exposure.

16. The sanitizing system of claim 13, further comprising a slidable drawer disposed in the cabinet housing above the compartment.

17. The sanitizing system of claim 16, wherein a floor of the slidable drawer is configured to allow transmission of ultraviolet radiation emitted by the UV radiation-producing device into the slidable drawer.

18. A method for sanitizing small objects, the method comprising:
opening an access door of a cabinet having an ultraviolet (UV) lamp housed therein;
placing a canister into a canister receiving structure of the cabinet through the access door, wherein the canister contains an object to be sanitized;
closing the access door and, using the UV lamp, irradiating the object in the canister with UV light for a selected duration, wherein the object is exposed to the UV light through a wall of the canister; and
increasing exposure of the object to the UV light by automatically reorienting the canister during the selected duration.

19. The method of claim 18, wherein the canister receiving structure comprises a powered roller assembly configured to automatically rotate the canister about a long axis thereof.

20. The method of claim 18, wherein the wall of the canister comprises a material having a transmittance of at least 90% with respect to UV light.

21. The method of claim 18, wherein the wall of the canister comprises an open mesh.
